(19) United States
(12) Patent Application Publication
(10) Pub. No.: US 2012/0283646 A1
(43) Pub. Date: Nov. 8, 2012

(54) DRUG DELIVERY SYSTEM, DRUG DELIVERY DEVICE, NEEDLE ASSEMBLY AND METHOD FOR PRIMING

(75) Inventors: Garen Kouyoumjian, Leamington Spa (GB); Robert Veasey, Leamington Spa (GB); Christopher Jones, Tewkesbury (GB); Catherine Anne Macdonald, Ashby-de-la-Zouch (GB); Stephen Gilmore, Leamington (GB); George Cave, Arborfield (GB)

(73) Assignee: SANOFI-AVENTIS DEUTSCHLAND GMBH, Frankfurt am Main (DE)

(21) Appl. No.: 13/498,372

(22) PCT Filed: Sep. 29, 2010
(86) PCT No.: PCT/EP2010/064423

§ 371 Date: Jul. 24, 2012

(30) Foreign Application Priority Data
Sep. 30, 2009 (EP) 09171763.7

(51) Int. Cl. A61M 5/315; A61M 5/32

(52) U.S. Cl. 604/192; 604/236

(57) ABSTRACT

A drug delivery system comprises a drug delivery device which comprises a housing and a cartridge. The drug delivery device has a proximal end and a distal end. The drug delivery system further comprises a needle assembly which is attachable to the drug delivery device. The needle assembly and the drug delivery device are configured such that the cartridge is moved in the proximal direction with respect to the housing when the needle assembly is attached to the drug delivery device.

### CROSS REFERENCE TO RELATED APPLICATIONS

The present application is a U.S. National Phase Application pursuant to 35 U.S.C. §371 of International Application No. PCT/EP2010/064423 filed Sep. 29, 2010, which claims priority to European Patent Application No. 09171763.7 filed on Sep. 30, 2009. The entire disclosure contents of these applications are herewith incorporated by reference into the present application.

### FIELD OF INVENTION

A drug delivery device is operable to deliver a drug from a cartridge via a cannula. A driving mechanism of the drug delivery device pushes a bung into the cartridge so that a predetermined dose is delivered.

### BACKGROUND

The dose may be variable or fixed. The drug delivery device or parts of it may be disposable or reusable. When the drug delivery device is assembled or parts of the drug delivery device, e.g. the cartridge, are exchanged, the parts of the driving mechanism may be not positioned in a predetermined position with respect to the cartridge or a housing of the drug delivery device. If the parts are positioned in the predetermined position, this ensures that a predetermined dose is delivered when the drug delivery device is used the first time after assembling. In other words, there may be an internal gap between parts of the drug delivery device which have to contact each other to ensure the delivery of the correct amount of the dose. The gap may be located between parts of the drive mechanism or between a part of the drive mechanism and e.g. the cartridge. The gap is a consequence of tolerances associated with all the assembled parts and the requirement not to preload the bung in the assembled device, because preloading would pressurise the drug in the cartridge.

A priming operation is performed to ensure that the parts of the driving mechanism are moved to their predetermined position with respect to the other parts.

### SUMMARY

Users who are not familiar with the drug delivery device may fail or incorrectly prime their drug delivery device before dispensing the first dose. If this occurs the user may inject the prime fluid or the correct dose of the drug may not be delivered in the first dose.

It is an aim of the present invention to provide a drug delivery system, a drug delivery device, a needle assembly and a method which prevent the user from dispensing the drug before priming.

For this aim a drug delivery system is provided which comprises a drug delivery device comprising a housing and a cartridge. The drug delivery device has a proximal end and a distal end. The drug delivery system further comprises a needle assembly which is attachable to the drug delivery device. The needle assembly and the drug delivery device are configured such that the cartridge is moved in the proximal direction with respect to the housing when the needle assembly is attached to the drug delivery device.

A method for priming a drug delivery device which comprises a housing, a piston rod and a cartridge with a bung comprises the following steps. A needle assembly is attached to the drug delivery device thereby moving the cartridge in the proximal direction with respect to the housing, so that the bung contacts the piston rod.

The needle assembly serves as priming tool. The drug delivery device is primed by means of the needle assembly during attachment of the needle assembly of the drug delivery device. The needle assembly is suitable for pushing the cartridge towards a driving mechanism of the drug delivery device, thereby closing a gap which may be located between the cartridge and the drive mechanism of the drug delivery device. The drug delivery system and the method prevent the user from injecting the prime fluid and encourage him or her to prime the drug delivery device correctly before use.

A drug delivery device is suitable for delivery of one or more doses of drugs contained in the cartridge. The doses may be fixed or variable. The drug delivery device may be of the injector type suitable for injecting. Alternatively, the drug delivery device may be used for pippetting into sample trays for example. The drug delivery device is reusable or disposable. In one embodiment the drug delivery device is a pen-type drug delivery device. The pen-type drug delivery device has an elongated form which has a tubular or non-tubular shape. One embodiment is shaped mainly as ellipsoid. An alternative embodiment is mainly cubical.

The housing comprises exterior or interior housing parts which are designed to enable safe, correct, and comfortable handling of the drug delivery device. The housing may be a unitary or a multipart component. The housing comprises a cartridge which is configured to contain the drug. The cartridge may be fixed or engaged with outer or inner part parts of the housing. A number of doses of the drug may be dispensed from the cartridge. An alternative embodiment of the cartridge is suitable for delivering a single dose.

The needle assembly comprises a cannula. The needle assembly is attached during the priming operation so that the drug can be delivered during the priming process when the cartridge is engaged with the housing. A priming dose may be delivered through the cannula when the cartridge is pushed towards the drive mechanism.

In one embodiment the needle assembly and the drug delivery device are configured such that a movement of the cartridge in the distal direction with respect to the housing is prevented when the needle assembly is attached to the drug delivery device. The attached needle assembly serves for locking the cartridge in a position in which it is in contact with the drive mechanism. In one embodiment the needle assembly comprises a separable part suitable for locking the cartridge in its position, the separable part remains on the drug delivery device when the needle assembly is detached after priming. One embodiment of the separable part comprises a needle unit which may be suitable for being used for the first injection after priming. This means that the needle assembly is suitable for priming the drug delivery device and for attaching a needle unit at the same time.

The drug delivery device may comprise a piston rod. The cartridge may comprise a bung. There is typically an internal gap between the distal end of the piston rod and the bung when the drug delivery device is assembled. This gap is a consequence of the tolerances associated with all the assembled parts and the requirement not to preload the bung in the assembled device, because preloading would pressurise the drug in the cartridge. The cartridge is moveable with respect to the piston rod so that the bung contacts the piston rod when the needle assembly is attached to the drug delivery device. Thereby the gap between the distal end of the piston rod and the bung is closed.

The needle assembly is suitable for pushing the cartridge during the priming step so that the piston rod abuts the bung. This step may come along with dispensing the drug from the cartridge through the cannula when the bung of the cartridge is moved towards the piston rod so that the bung is pushed into the cartridge thereby dispensing the drug.

For the aim of preventing the user from dispensing the drug before priming, the drug delivery device comprises a housing, a cartridge and a guard member which is attached to a distal part of at least one of the housing and the cartridge. The guard member is configured to be detached during a priming process or after a priming process.

The presence of the guard member which is mounted to the drug delivery device clearly indicates that the drug delivery device has not been primed yet. One embodiment of the guard member prevents the use of the drug delivery device for injection. The guard member may be mounted to the distal end of the drug delivery device so that a needle unit which is suitable for injection cannot be attached to the drug delivery device. Alternatively the guard member may block the drive mechanism. One embodiment of the guard member is essentially cylindrically shaped.

One embodiment of the guard member comprises an engagement means which is releasably engaged with a coupling means of at least one of the housing and the cartridge. In one embodiment the coupling means comprises openings of the housing, and the engagement means comprises fingers which extend radially inwards in the openings of the housing. The engagement means can be decoupled from the coupling means during the priming process which means during the attachment of the needle assembly.

A needle assembly is configured to be attached to a drug delivery device, the needle assembly comprising a needle hub which is attachable to the drug delivery device and a needle unit which comprises a cannula. The needle unit is releasably coupleable to the needle hub.

The needle assembly serves for attaching the needle unit and the needle hub to the drug delivery device. The needle hub serves as a connection piece between the needle unit and the drug delivery device. The needle unit is detachable from the needle hub which remains connected with the drug delivery device. In one embodiment the needle assembly provides the needle unit which is releasably coupled to the needle hub. In an alternative embodiment the needle unit is being mounted to the needle hub during the attachment process. The needle unit is suitable for delivering the drug through the cannula.

In one embodiment the needle hub and needle unit are threadedly connected. In other words, the needle unit has a thread, preferably an internal thread, which engages with a thread, preferably an external thread, of the needle hub.

In one embodiment the needle assembly comprises a cover which at least partly covers the needle unit and the needle hub, the cover being suitable for holding the needle unit and the needle hub during the attachment of the needle hub. The cover serves as a tool for attaching the needle unit and the needle hub to the drug delivery device. In one embodiment the cover protects the needle unit and the needle hub which are located at least partly inside the cover.

One embodiment of the needle assembly comprises a drug trapping means suitable for trapping a fluid which is dispensed through the cannula. The drug trapping means may serve as an absorbing means. Such an absorbing means may comprise a sponge or fluid absorbing granules.

The needle assembly can be attached to a drug delivery device comprising a housing and a cartridge which moves in the proximal direction with respect to the housing, when the needle assembly is attached. In other words, the needle assembly serves as a priming tool. The drug delivery device is primed during attachment of the needle unit and the needle hub by means of the needle assembly.

In one embodiment the needle hub comprises an attachment means which is configured to engage with the drug delivery device. One embodiment of the attachment means is designed as a snapping means. The needle assembly may be attachable to a drug delivery device as described above. The needle assembly can be used in conjunction with a disposable fixed or variable drug delivery device, e.g. a pen injector. The drug delivery device may have a push-pull mechanism for setting and dispensing the dose. The needle assembly is applicable, though not limited, to disposable fixed dose injectors, in which the usability issues associated with the set-up of new devices may be encountered.

One embodiment of the needle assembly comprises a decoupling means which is configured to detach the guard member during attachment of the needle assembly to the drug delivery device. The decoupling means is suitable for disconnecting the guard member. In one embodiment the guard member is disconnected when the needle unit and the needle hub are attached to the drug delivery device. The disengagement means may be configured to bring the engagement means of the guard member out of engagement with the coupling means when the needle assembly is attached to the drug delivery device. In one embodiment the disengagement means comprises fingers which are configured to couple the needle hub to the drug delivery device. This means that mounting the needle unit and the needle hub results in decoupling the guard member. The fingers may have snapping means which are connectable with the drug delivery device.

One embodiment of the needle assembly comprises a holding means which is configured to at least partly receive the guard member when the needle assembly is attached to the drug delivery device. The guard member is inserted into the needle assembly during attachment of the needle unit and the needle hub. Preferably the holding means is suited for holding the detached guard member. In one embodiment the cover comprises the holding means which may be formed as a cavity of the cover. Thus the guard member remains in the cover after detachment.

Other features will become apparent from the following detailed description when considered in conjunction with the accompanying drawings.

The term “drug”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(w-carboxyheptadecanoyl)-des(B30) human insulin and B29-N-(w-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

### BRIEF DESCRIPTION OF THE FIGURES

FIG. 1 shows a three-dimensional cut-away view of an embodiment of a drug delivery device before first use and an embodiment of a needle assembly suitable for priming the drug delivery device.

FIG. 2 shows a three-dimensional view of an embodiment of a guard member which is mounted to the housing of the drug delivery device.

FIG. 3 shows a three-dimensional view of the needle assembly comprising a seal before use.

FIG. 4 shows a three-dimensional view of the needle assembly after removing the seal.

FIG. 5 shows a cross-sectional view of the drug delivery device and the needle assembly before use.

FIG. 6 shows a cross-sectional view of the drug delivery device and the needle assembly, wherein the needle assembly is attached to the drug delivery device.

FIG. 7 shows a cross-sectional view of the drug delivery device and the needle assembly, wherein the needle assembly is detached from the drug delivery device.

### DETAILED DESCRIPTION

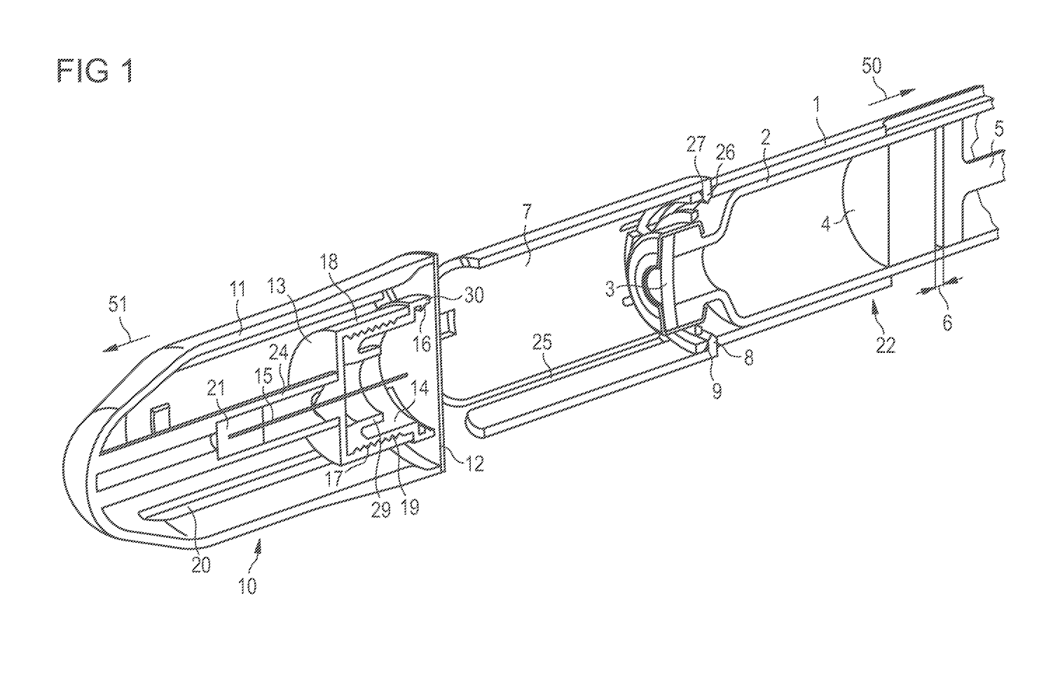

FIG. 1 shows a three-dimensional cut-away view of an embodiment of a drug delivery device 22 before first use and a needle assembly 10 suitable for priming the drug delivery device 22.

The drug delivery device 22 comprises a housing 1 having a distal part and a proximal part. The distal end of the device 22 is indicated by arrow 51, which refers to that end of the drug delivery device 22 which is closest to a dispensing end of the drug delivery device 1. The proximal end of the device 22 is indicated by arrow 50 referring to that end of the device 22 which is furthest away from the dispensing end of the device 22.

The drug delivery device 22 may be designed for injection of fixed doses. Alternatively, the drug delivery device may be designed for injection of variable doses.

A cartridge 2 which contains the drug to be delivered is located in the distal part of the housing 1 which serves as a cartridge holder. In a fixed dose drug delivery device the cartridge 2 contains a number of doses of a fluid drug. In one embodiment the cartridge merely contains a single dose of drug. The cartridge 2 may be used with drug delivery devices 22 that deliver various fluid drugs, e.g. glucagon-like peptide-1 (GLP-1), heparin.

The distal end of the cartridge 2 has an opening which is covered by a septum 3. The septum 3 can be punctured by a cannula through which the drug is expelled. A bung 4 is located in the proximal part of the cartridge 2. The bung 4 is moveable along the inner side wall of the cartridge 2 in the distal direction.

In one embodiment the cartridge 2 is disposable, which means that the cartridge 2 can be removed from the housing 1 and a new cartridge 2 can be fit into the housing 1. In one embodiment the distal and the proximal part of the housing 1 can be disassembled for the purpose of replacing the cartridge 2. In another embodiment the distal part of the housing 1 which serves as a cartridge holder is permanently attached to the proximal part of the housing 1. The cartridge may be removed from or inserted to the housing 1 through a distal opening of the housing 1 during replacement. In an alternative embodiment the distal part of the housing 1 is also removable, which means that the cartridge 1 and the distal part of the housing 1 are replaceable.

The drug delivery device 22 further comprises a drive mechanism (not completely shown) having a piston rod 5 located inside the housing 1. The drive mechanism is suitable for moving the bung 4 during injection so that a predetermined amount of drug can be expelled from the cartridge 2 through the cannula. The piston rod 5 is moveable with respect to the housing 1 and suitable for pushing the bung 4 in the distal direction along the inside wall of the cartridge 2 when the piston rod 5 is moved in the distal direction with respect to the housing 1. The dose to be delivered is set by a dose setting mechanism (not shown). The dose is delivered in response to the movement of a button member (not shown) after setting the dose.

During normal operation (not shown in FIG. 1) a needle unit with a cannula is attached to the distal end of the drug delivery device so that the cannula punctures the septum 3. The distal end of the piston rod 5 abuts the bung 4. A dose of the drug is delivered through the cannula when the piston rod 5 is moved by a given distance in the distal direction with respect to the housing 1, thereby pushing the bung 4 by the given distance in the distal direction. If the piston rod 5 would not abut the bung 4 the correct volume of the dose may not be delivered.

However, there is typically an internal gap 6 between the distal end of the piston rod 5 and the bung 4 when the drug delivery device is assembled. The piston rod 5 may not lie against the bung 4 before first use of the drug delivery device 22. In this case the distance travelled by the piston rod 5 would not correspond with the distance travelled by the bung 4 so that an incorrect dose would be delivered.

This gap 6 is a consequence of the tolerances associated with all the assembled parts and the requirement not to preload the bung 4 in the assembled drug delivery device. FIG. 1 shows the gap 6 between the distal end of the piston rod 5 and the bung 4. Before first use the piston rod 5 has to abut against the bung 4. This priming step prevents the delivery of an incorrect volume of the dose during first use of the drug delivery device 22.

Furthermore, the drug delivery device 22 comprises a guard member 7 which is mounted to the distal end of the housing 1. In an alternative embodiment (not shown) the guard member 7 is mounted to the distal end of the cartridge 2. The guard member 7 prevents the attachment of a disposable needle unit, which is used for injecting the drug to a person's or an animal's body, to the cartridge 2. In other words, if the guard member 7 is attached to the housing 1 the drug delivery device 22 cannot be used for injection.

This embodiment of the guard member 7 has a mainly tubular shape and surrounds the distal end of the cartridge 2 and the septum 3. The guard member comprises an engagement means 8 which releasably engages with a coupling means 9 which is located at the distal part of the housing 1. In an alternative embodiment (not shown) the coupling means are positioned at the cartridge 2. In the embodiment shown in FIG. 1 the engagement means 8 comprises fingers 26 which extends radially inwards in openings 27 of the housing. The guard member 7 comprises slots 25 so that the guard member 7 is moveable along splines 20 in the needle assembly 10 as described later.

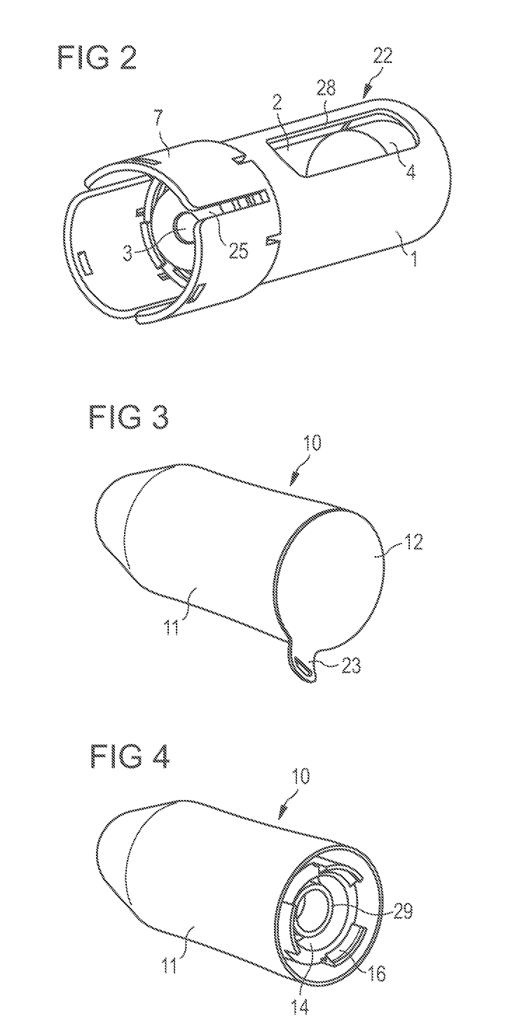

FIG. 2 shows a three-dimensional view of the guard member 7 mounted to the housing 1 of the drug delivery device 22. Furthermore FIG. 4 shows a window 28 in the distal part of the housing 1, the window being formed as cut-out. The cartridge 2 is visible through the window 28. If the cartridge 2 is made of a transparent material, e.g. glass, the position of the bung 4 in the cartridge 4 is visible, which indicates the liquid level of the drug inside the cartridge 2.

The guard means 7 prevents the user from dispensing the drug until the drug delivery device 22 has been primed. This ensures that the user is forced to undertake a priming operation prior to delivering the first dose of drug.

The presence of the guard member indicates that the drug delivery device has not been used yet and that priming is necessary before first use.

The guard member 7 is mounted externally to the distal end of the housing 1 such that it cannot move relative to the housing 1 and prevents fitment of a needle unit which is used for injection to the distal end of the cartridge 2. In one embodiment the guard member 7 prevents fitment of a standard A type needle unit, which may be used on many pen injectors.

Furthermore, FIG. 1 shows a priming needle assembly 10 which is separate to the drug delivery device 22. The needle assembly 10 comprises a cover 11 which may be formed as an essentially tubular cap which has a cavity. The cavity of the cover 11 is closed with a sterile seal 12.

FIG. 3 shows a three-dimensional view of the needle assembly 10 before use, wherein the cover 11 is closed with the sterile seal 12 which comprises an ear 23 suitable for being gripped by a user while removing the seal 12.

Referring back to FIG. 1, the needle assembly 10 also comprises a needle unit 13 which is releasably connected with a needle hub 14, the needle unit 13 and the needle hub 14 being located inside the cavity of the cover 11. The cover 11 covers the needle unit 13 and the needle hub 14. The seal 12 is affixed to the proximal end of the cover 11 and serves to encapsulate the needle unit 13 and the needle hub 14 within the cover 11. This allows for the contents of the cover 11 to be sterilized prior to delivery to the user.

The cover 11 holds the needle unit 13 and the needle hub 14 and serves as a tool for attaching the needle unit 13 and the needle hub 14. A needle holding means are suitable for holding the needle unit 13 and the needle hub 14 in a position along a main axis of the needle assembly 10. The needle holding means are formed as splines 20 suitable for holding the needle unit 13 and the needle hub 14 along the main axis so that there is an interspace between the needle unit 13 mounted on the needle hub 14 and the inner side wall of the cover 11. The splines 20 extend along the inner side wall of the cover 11. They are arranged in such a way, that the guard member 7 can be inserted into the cavity of the cover 11, while the needle unit 13 and needle hub 14 are located inside.

The cover 11 comprises a stopping means which is suitable for preventing a movement of the needle unit 13 and the needle hub 14 in the distal direction with respect to the cover 11. In this embodiment the stopping means is designed as stopping cylinder 24 extending from the distal end of the cover 11 along the main axis of the needle assembly 10. The proximal end of the stopping cylinder 24 abuts the needle unit 13 or is positioned close to the needle unit 13. Nevertheless the distal part of the cannula 15 of the needle unit lies inside the stopping cylinder 24.

The needle assembly 10 comprises a drug trapping means 21 which is suitable for trapping a drug delivered during the priming process. In this embodiment the drug trapping means 21 is formed as sponge insert which is positioned in the stopping cylinder 24. In one embodiment the sponge insert is fixed within the cover 11.

The needle unit 13 comprises a cannula 15 through which the drug is dispensed. One embodiment of the needle unit 13 is used only during the priming process. One embodiment of the needle unit 13 is used for the first injection after the priming process. Usually the needle unit 13 is disposed after injection and a new needle unit is attached before injection of the next dose for sterility reasons. While the needle unit 13 is usually removed after each injection the needle hub 14 remains on the cartridge 2.

The needle hub 14 can be connected with the distal end of the housing 1. Once the needle hub 14 has been attached to the housing 1 the needle hub 14 remains on the housing 1 during the life time of the cartridge 2. The needle units which are used for injection are attached to the needle hub 14 before injection and removed from the needle hub 14 after injection.

In one embodiment the needle hub 14 is attached to the housing 1 permanently. In an alternative embodiment the needle hub 14 is attached directly to the cartridge 2 and disposed with the cartridge 2 when depleted, which requires a new needle assembly each time.

The needle hub 14 is positioned inside the cover 11 along the main axis of the needle assembly 10 and is essentially cylindrical in design with an external helical thread 17 for mounting of the disposable needle units 13. A pushing cylinder 29 is located inside the cylindrical needle hub 14. This cylinder 29 pushes against the cartridge when the needle assembly 10 is attached thereby pushing the cartridge 2 towards the piston rod 5. A hole at the distal end of the needle hub 14 allows insertion of the cannula 15 through the internal cylinder 29 inside the needle hub 14. The needle hub 14 comprises an attachment means configured to engage with the drug delivery device. In one embodiment the needle hub 14 comprises snap engagement means 16 located at the proximal end of the needle hub 14, the snap engagement means 16 are suitable for mounting the needle hub 14 to the distal end of the cartridge 2. The proximal end of the snap engagement means 16 are formed as arms 30 which are suitable for decoupling the guard member 7 from the housing 1 of the drug delivery device 22. The needle unit 13 and needle hub 14 is mounted on the holding splines 20 within the cover 11 such that relative axial movement is permitted but relative rotational movement is not. In one embodiment the needle unit 13 has trenches in which the splines 20 engage.

The needle unit 13 comprises an attachment mount 18 which is suitable for releasably coupling with the needle hub 14. The attachment mount 18 which may be cylindrically formed comprises an internal thread 19 located along the inside of the attachment mount 18. The internal thread 19 is in engagement with the external thread 17 of the needle hub 14.

FIG. 4 shows a three-dimensional view of the needle assembly 11 after removing the seal 12, wherein the needle hub 14 with the snap engagement means 16 inside the cover 11 is visible.

Referring back to FIG. 1, the needle unit 13 is mounted along the main axis of the needle assembly 12. The distal end of the cannula 15 is located within the drug trapping means 21 while the proximal end is located within the attachment mount 18 of the needle unit.

When the drug delivery device 22 and the needle assembly 10 are delivered to the user, the drug delivery device 22 comprising the guard member 7 is supplied separately to the needle assembly 10. The drug delivery device 22 has to be primed before first use, which means the piston rod 5 has to abut on the bung 4.

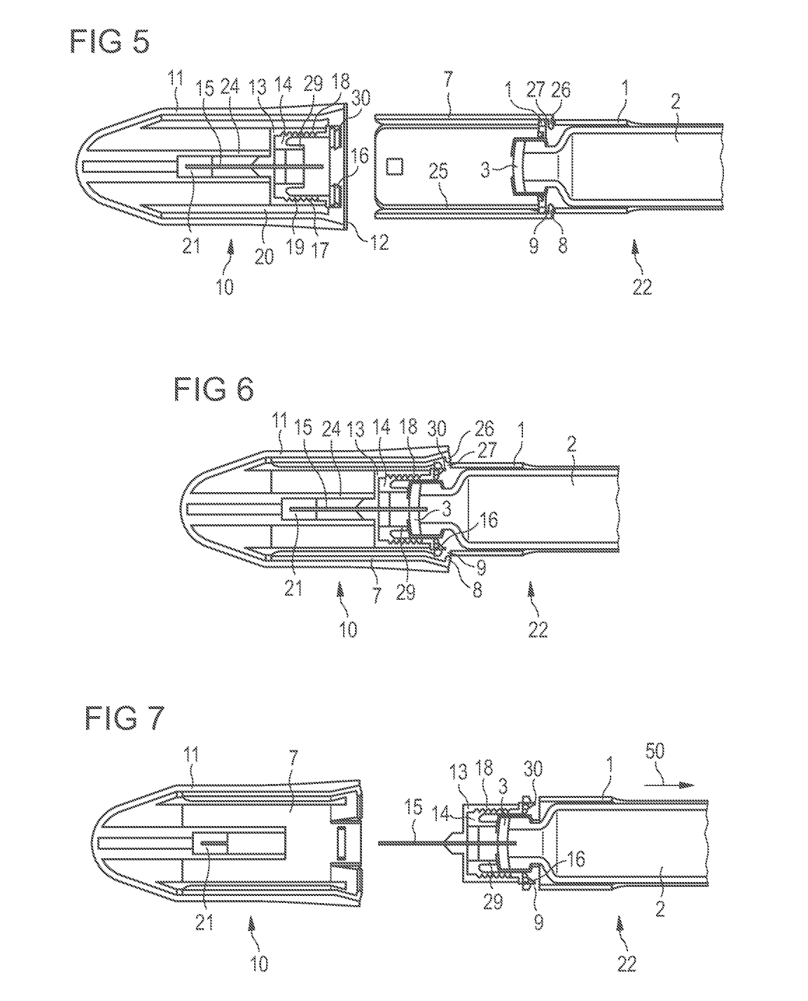

FIG. 5 shows a cross-sectional view of the drug delivery device and the needle assembly before first use. The reference numerals are related to the same features as in FIG. 1.

When the drug delivery device 22 and the needle assembly 10 are provided, which means delivered to the user, the drug delivery device 22 is supplied separately to the needle assembly 10. In this state the guard member 7 prevents fitment of a needle unit other than the needle unit 13 mounted to the needle hub 14 which are provided in the needle assembly 10. This in turn prevents the dispensing of a dose of drug from the drug delivery device 22 to the user.

The priming operating is performed by means of the needle assembly 10. The drug delivery device 22 is primed when the needle assembly 10 is attached to the drug delivery device 22, which will now be described.

Prior to first use the user removes the sterile seal 12 located on the cavity of the cover 11, thereby exposing the needle hub 13 inside the cover 11 (as shown in FIG. 4).

The cover 11 is pressed axially towards the distal end of the housing 1 so that the guard member 7 is inserted into the cavity of the cover 11. The cover 11 fits over the guard member 7 and the splines 20 in the cover 11 pass axially into the slots 25 in the guard member 7.

In addition, axial movement of the cover 11 towards the proximal end of the drug delivery device 22 forces the proximal end of the cannula 15 to pierce the septum 3, bringing the drug into communication with the drug trapping means 21 through the cannula 15. Further axial movement cause the internal cylinder 29 of the needle hub 14 to abut the distal end of the cartridge 2. Continued axial motion forces the cartridge 2 proximally back against the drive mechanism until the bung 4 abuts the piston rod 5 of the drive mechanism. As the cartridge 2 continues to move axially relative to the now stationary bung 4 that is now abutting the drive mechanism, the priming drug is forced out of the cartridge 2 through the cannula 15 into the drug trapping means 21, wherein the sponge insert absorbs the drug. The drug which is dispensed during priming is contained in the drug delivery device 22 and controlled, which ensures that the priming dose cannot be accidentally injected into the user. The drug is safely contained within an appropriately designed receptacle.

FIG. 6 shows a cross-sectional view of the drug delivery device 22 and the needle assembly 10, wherein the needle assembly 10 is attached to the drug delivery device 22.

At the end of the axial travel of the cover 11 the snap engagement features 16 of the needle hub 14 engage permanently with the housing 1. Simultaneously arms 30 of the needle hub 14 pass underneath the snap engaging finger 26 connecting the guard member 7 to the housing 1 and displace them radially, disengaging the guard means 7 from the housing 1. This ensures that the guard member 7 cannot be removed from the drug delivery device 22 until the priming dose has been fully dispensed and the needle hub 14 is secured to the drug delivery device.

Subsequently the cover 11 is pulled axially in the distal direction off the housing

FIG. 7 shows a cross-sectional view of the drug delivery device and the needle assembly, wherein the cover 11 is removed from drug delivery device 22 after priming.

The guard member 7 and the sponge insert which serves as drug trapping means 21 are retained in the cover 11. The guard member is positioned at least partly in the cavity of the cover 11, the cavity serving as holding means suitable for holding the guard member 7. The guard member 7 may be connected with the cover e.g. by clips or by friction. The sponge insert may be connected with the cover 11 e.g. by friction, glue or clip.

The needle hub 14 with the mounted needle unit 13 is retained on the housing 1. Removal of the cover 11 exposes the needle unit 13 fitted to the needle hub 14 and in communication with the drug in the cartridge 2 and thus leaves the drug delivery device ready 22 for injection of the first dose to the user. The needle hub 14 remains permanently attached to housing 1, ready for attachment of further needles units as necessary.

In summary, the drug delivery device 22 and the needle assembly 10 have a relatively low number of components compared to a conventional drug delivery device. The device and the needle assembly eliminate the risk of accidental injection of the priming dose. The needle assembly and the guard member can be fitted to any drive mechanism and any drug delivery device, which makes it suitable for a wide range of drug delivery devices.

Other implementations are within the scope of the claims. Elements of different embodiments may be combined to form implementations not specifically described herein.

1-20. (canceled)

21. A drug delivery system comprising
a drug delivery device which comprises a housing, a piston rod and a cartridge comprising a bung and being moveable with respect to the piston rod, the drug delivery device having a proximal end and a distal end, and
a needle assembly which is attachable to the drug delivery device, the needle assembly and the drug delivery device being configured such that the cartridge is moved in the proximal direction with respect to the housing so that the bung contacts the piston rod when the needle assembly is attached to the drug delivery device.

22. The drug delivery system according to claim 21, wherein the needle assembly and the drug delivery device are configured such that a movement of the cartridge in the distal direction with respect to the housing is prevented when the needle assembly is attached to the drug delivery device.

23. The drug delivery device comprising a housing, a piston rod, a cartridge and a guard member which is attached to a distal part of at least one of the housing and the cartridge, the guard member being configured to be detached during a priming process or after a priming process wherein during the priming process the cartridge is moved in the proximal direction with respect to the housing, so that a bung of the cartridge contacts the piston rod.

24. The drug delivery device according to claim 23, wherein the guard member comprises an engagement means which is releasably engaged with a coupling means of at least one of the housing and the cartridge.

25. The drug delivery device according to claim 24, wherein the coupling means comprises openings of the housing, and wherein the engagement means comprises fingers which extend radially inwards in the openings of the housing.

26. A needle assembly being attachable to a drug delivery device according to claim 3, the needle assembly comprising a needle hub being attachable to the drug delivery device and a needle unit comprising a cannula, the needle unit being releasably coupleable to the needle hub.

27. The needle assembly according to claim 26, wherein the needle assembly comprises a cover, which at least partly covers the needle unit and the needle hub, the cover being suitable for holding the needle unit and the needle hub during the attachment of the needle hub.

28. The needle assembly according to claim 26, wherein the needle hub and needle unit are threadedly connected.

29. The needle assembly according to claim 26, comprising a drug trapping means suitable for trapping a fluid which is dispensed through the cannula.

30. The needle assembly according to claim 26, wherein the needle assembly is attachable to a drug delivery device comprising a housing and a cartridge which moves in the proximal direction with respect to the housing, when the needle assembly is attached.

31. The needle assembly according to claim 30, wherein the needle hub comprises an attachment means configured to engage with the drug delivery device.

32. The needle assembly according to claim 26, comprising a decoupling means which is configured to detach the guard member during attachment of the needle assembly to the drug delivery device.

33. The needle assembly according to claim 6, comprising a holding means which is configured to at least partly receive the guard member when the needle assembly is attached to the drug delivery device.

34. The needle assembly according to claim 33, wherein the holding means is suited for holding the detached guard member.

35. The needle assembly according to claim 33, wherein the cover comprises the holding means.

36. The needle assembly according to claim 33, comprising a disengagement means configured to bring the engagement means of the guard member out of engagement with the coupling means when the needle assembly is attached to the drug delivery device.

38. The needle assembly according to claim 36, wherein the disengagement means comprises fingers which are configured to couple the needle hub to the drug delivery device.

39. A method for priming a drug delivery device which comprises a housing, a piston rod and a cartridge with a bung, the method comprising attaching a needle assembly to the drug delivery device thereby moving the cartridge in the proximal direction with respect to the housing, so that the bung contacts the piston rod.
